(19) United States
(12) Patent Application Publication
(10) Pub. No.: US 2016/0258025 A1
(43) Pub. Date: Sep. 8, 2016

(54) PREDICTING PATIENT RESPONSIVENESS TO IMMUNE CHECKPOINT INHIBITORS

(71) Applicants: THE REGENTS OF THE UNIVERSITY OF CALIFORNIA, Oakland, CA (US); ADAPTIVE BIOTECHNOLOGIES CORP., SEattle, WA (US)

(72) Inventors: Mark KLINGER, Seattle, WA (US); Malek FAHAM, Seattle, WA (US); Martin MOORHEAD, Seattle, WA (US); Lawrence FONG, Oakland, CA (US)

(21) Appl. No.: 15/030,166

(22) PCT Filed: Oct. 17, 2014
(86) PCT No.: PCT/US2014/061260

§ 371 Date: Apr. 18, 2016

Related U.S. Application Data
(60) Provisional application No. 61/892,711, filed on Oct. 18, 2013.

(51) Int. Cl. C12Q 1/68

(52) U.S. Cl. CPC C12Q 1/6886; C12Q 2600/106

(57) ABSTRACT

The invention is directed to a method of predicting clinical response of a patient to treatment of a cancer by an immune checkpoint pathway inhibitor, such as an anti-CTLA-4 or anti-PD-1 antibody binding compound. In one aspect the method comprises generating pre- and post-treatment clonotype profiles, determining a number of clonotypes that decrease in frequency between the first and second clonotype profiles, and predicting a lack of responsiveness in the patient to the treatment whenever the number of clonotypes that decrease in frequency is greater than a predetermined value.

### CROSS-REFERENCE

This application claims the benefit of U.S. Provisional Application No. 61/892,711, filed Oct. 18, 2013, which application is incorporated herein by reference.

### BACKGROUND OF THE INVENTION

Cellular immune responses are controlled by stimulatory and inhibitory pathways that act in concert under normal conditions. The ability of cancer cells to evade destruction by immune surveillance has been attributed to the aberrant production of compounds, perhaps by tumor cells themselves or by cells in their vicinity, that trigger immune inhibitory pathways, that is, immune checkpoint pathways, thereby permitting tumors to persist and spread, e.g. Pardoll, Nature Reviews Cancer, 12: 252-264 (2012); Gelao et al, Toxins, 6: 914-933 (2014). This observation has provided the basis for a promising new approach to cancer treatment: if an immune inhibitory pathway turned on by a tumor could be reversed, then immune surveillance may be reactivated and tumors destroyed by a cytotoxic immune response against tumor cells. Initial tests of this approach have shown remarkable success and numerous clinical trials of immune checkpoint pathway inhibitors have been initiated, e.g. Pardoll (cited above); Gelao et al (cited above). Targets of such inhibitors have included the CTLA-4 receptor, PD-1 receptor, and the PD-1 ligand, PDL-1, as well as other inhibitory pathway components, e.g. Pardoll (cited above); Gelao et al (cited above). Although many patients seem to benefit from the new treatments, results are not uniform for all patients; thus, there is a critical need for developing biomarkers that permit identification of patients that will benefit from a particular treatment.

The circumstances of the CTLA-4 inhibitory pathway are representative of this need. Two proteins on the surface of T cells—CD28 and cytotoxic T-lymphocyte antigen 4 (CTLA-4)—play important roles in the regulation of immune activation and tolerance. CD28 provides positive modulatory signals in the early stages of an immune response, while CTLA-4 signaling inhibits T-cell activation, particularly during strong T-cell responses. CTLA-4 blockade using CTLA-4 inhibitors, such as anti-CTLA-4 monoclonal antibodies, has great appeal because suppression of inhibitory signals results in the generation of an antitumor T-cell response. Both clinical and preclinical data indicate that CTLA-4 blockade results in direct activation of CD4+ and CD8+ effector cells, and anti-CTLA-4 monoclonal antibody therapy has shown promise in a number of cancers, particularly melanoma, Leach et al, Science, 271: 1734-1736 (1996); Wolchok et al, Oncologist, 13: Suppl 4: 2-9 (2008) Like many targeted therapies, responsiveness to CTLA-4 inhibition depends on a wide range of factors and is not uniform among patients; nonetheless, a fraction of all patients suffer significant adverse reactions to such treatment, e.g. Lipson et al, Clinical Cancer Research, 17(22): 6958-6962 (2011).

Recently, diagnostic and prognostic applications have been proposed that use large-scale DNA sequencing as the per-base cost of DNA sequencing has dropped and sequencing techniques have become more convenient, e.g. Welch et al, Hematology Am. Soc. Hematol. Educ. Program, 2011: 30-35; Cronin et al, Biomark Med., 5: 293-305 (2011); Palomaki et al, Genetics in Medicine (online publication 2 Feb. 2012). In particular, profiles of nucleic acids encoding immune molecules, such as T cell or B cell receptors, or their components, contain a wealth of information on the state of health or disease of an organism, so that diagnostic and prognostic indicators based on the use of such profiles are being developed for a wide variety of conditions, Faham and Willis, U.S. patent publication 2010/0151471; Freeman et al, Genome Research, 19: 1817-1824 (2009); Boyd et al, Sci. Transl. Med., 1(12): 12ra23 (2009); He et al, Oncotarget (Mar. 8, 2011). Recently, such techniques have been used to study the effects CTLA-4 inhibitors have on patient T cell repertoires and have shown that such inhibitors stimulate significant clonotype turnover in T cell receptor repertoires, e.g. Cha et al, J. Clin. Oncol., 31, 2013 (suppl; abstract 3020).

In view of the above, it would be highly useful for cancer treatment employing CTLA-4 inhibitors if readily measured patterns of T cell receptor representation in clonotype profiles from patient samples could be used to provide prognostic information regarding a patient's responsiveness to such treatment.

### SUMMARY OF THE INVENTION

The present invention is drawn to methods for using information in T cell receptor clonotype profiles of patients undergoing cancer therapy with a checkpoint inhibitor, such as a CTLA-4 inhibitor, to determine whether such patients will be responsive to, and benefit from, such therapy. The invention is exemplified in a number of implementations and applications, some of which are summarized below and throughout the specification.

In one aspect, the invention is directed to a method of predicting clinical response of a patient to treatment of a cancer by an immune checkpoint pathway inhibitor comprising the steps of: (a) generating a first clonotype profile from recombined T cell receptor genes or nucleic acids transcribed therefrom from a first patient sample taken before treatment by an immune checkpoint pathway inhibitor; (b) generating a second clonotype profile from recombined T cell receptor genes or nucleic acids transcribed therefrom from a second patient sample taken during or after treatment by an immune checkpoint pathway inhibitor; (c) determining a number of clonotypes that decrease in frequency between the first and second clonotype profiles; and (d) predicting a lack of responsiveness in the patient to the treatment whenever the number of clonotypes that decrease in frequency is greater than a predetermined value.

In another aspect, the invention is directed to a method of predicting clinical response of a patient to treatment of a cancer by an immune checkpoint pathway inhibitor that is a CTLA-4 inhibitor.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the invention.

### BRIEF DESCRIPTION OF THE DRAWINGS

The novel features of the invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIG. 1A shows data indicating that prostate cancer patients with lower overall survival have (on average) a larger number of clonotypes with reductions in frequency between the initiation of therapy and those with higher overall survival (on average) have a lower number of clonotypes with reductions in frequency.

FIG. 1B shows data indicating that melanoma cancer patients with lower overall survival have (on average) a larger number of clonotypes with reductions in frequency between the initiation of therapy and those with higher overall survival (on average) have a lower number of clonotypes with reductions in frequency.

FIGS. 2A-2B-2C show a two-staged PCR scheme for amplifying TCRβ genes.

FIG. 3A illustrates details of determining a nucleotide sequence of the PCR product of FIG. 2C. FIG. 3B illustrates details of another embodiment of determining a nucleotide sequence of the PCR product of FIG. 2C.

FIG. 4 illustrates data showing the influence of CTLA-4 blockade on TCR diversity.

FIGS. 5A-5B illustrate data comparing changes in TCR repertoire of untreated individual and treated individual over same time intervals, where the treated individual's time interval spanned treatment with CTLA-4 inhibitor.

FIG. 5C illustrates data showing repertoire turnover induced by CTLA-4 blockade.

FIG. 6 illustrates data showing numbers of clones with modified abundance induced by CTLA-4 blockade of untreated control, prostate cancer patients and melanoma cancer patients.

FIGS. 7A-7D illustrates data showing antigen specificity of specific T cell clonotypes. FIG. 7A shows that gated populations indicate CD8+ CMV pp65 (495-404) tetramer− (rectangle 702) and tetramer+ (oval 700) populations sorted by flow cytometry and assessed for TCRβ repertoire sequencing from a clinical responder. FIG. 7B shows that the frequency (log 10) of each clone identified following TCRβ repertoire sequencing of the sorted tetramer+ and tetramer− CD8+ T cells are shown. The three clones indicated in black (704) are those deemed antigen-specific based on fold-enrichment in tetramer+ versus tetramer− T cells and absolute frequency in tetramer+ cells. FIG. 7C shows that TCRβ clone frequencies (log 10) at baseline (week 0) and post-treatment (week 16). The three clones identified in FIG. 7C are indicated in black (706). FIG. 7D shows that frequencies of the three CMV-specific clones identified in (FIG. 7B) and (FIG. 7C) at baseline (week 0) and at various time points following anti-CTLA-4 treatment.

### DETAILED DESCRIPTION OF THE INVENTION

The practice of the present invention may employ, unless otherwise indicated, conventional techniques and descriptions of molecular biology (including recombinant techniques), bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; and Molecular Cloning: A Laboratory Manual (all from Cold Spring Harbor Laboratory Press); and the like.

In one aspect, the invention is directed to methods for prognosing responsiveness of a patient to treatment with an anti-cancer agent comprising at least one immune checkpoint pathway inhibitor, such as a CTLA-4 inhibitor. Such methods rely on changes in clonotype profiles of a patient brought about by such treatment. In accordance with the invention, methods are provided for generating successive clonotype profiles where at least one is from a sample taken at or prior to initiation of therapy and where at least one is taken during or after initiation of therapy, for example, in some embodiments, from one week to six months after initiation of therapy, or, in other embodiments, from two weeks to three months after initiation of therapy. After such clonotype profiles are obtained the frequencies of matching clonotypes are compared between the successive profiles. Whenever the number of such clonotype frequencies that decrease from the first measurement to a successive measurement is above a predetermined value, the patient is unlikely to respond to treatment with the immune checkpoint inhibitor being employed. As noted in the definition section, the term “clonotype profile” refers (in some embodiments) to a tabulation of nucleotide sequence frequencies, where the nucleotide sequences encode immune receptor molecules or portions thereof, such as, a CDR3 region of a TCR chain. In some applications, the method of the invention may be used with a sequence of immune checkpoint pathway inhibitors until one is found that a patient responds to. That is, if a patient is treated with an initial immune checkpoint pathway inhibitor and the method of the invention indicates lack of responsiveness, another immune checkpoint pathway inhibitor, for example inhibiting a separate pathway, may be tried, after which the method of the invention can be used to test for responsiveness again. Such a trial and error process may continue until an effective immune checkpoint pathway inhibitor is found for the patient.

Accordingly, in some embodiments, a method of treating a patient suffering from a cancer may comprise the steps of: (a) generating a first clonotype profile from recombined T cell receptor genes or nucleic acids transcribed therefrom from a first patient sample taken before a first anti-cancer treatment comprising a first immune checkpoint pathway inhibitor; (b) generating a second clonotype profile from recombined T cell receptor genes or nucleic acids transcribed therefrom from a second patient sample taken during or after the first anti-cancer treatment; (c) determining a number of clonotypes that decrease in frequency between the first and second clonotype profiles; and (d) switching from the first anti-cancer treatment to a second anti-cancer treatment comprising a second immune checkpoint pathway inhibitor different from the first immune checkpoint pathway inhibitor whenever the number of clonotypes that decrease in frequency is greater than a predetermined value.

In other embodiments, the invention includes a method of predicting clinical response of a patient to treatment of a cancer by an immune checkpoint pathway inhibitor, such as a PD-1 inhibitor or a CTLA-4 inhibitor, which comprises the following steps: (a) generating a first clonotype profile from recombined T cell receptor genes or nucleic acids transcribed therefrom from a first patient sample taken before treatment by an immune checkpoint pathway inhibitor, such as a PD-1 inhibitor or a CTLA-4 inhibitor; (b) generating a second clonotype profile from recombined T cell receptor genes or nucleic acids transcribed therefrom from a second patient sample taken during or after treatment by the immune checkpoint pathway inhibitor; (c) determining a number of clonotypes that decrease in frequency between the first and second clonotype profiles; and (d) predicting a lack of responsiveness in the patient to the treatment whenever the number of clonotypes that decrease in frequency is greater than a predetermined value.

In part the invention is based on the recognition and appreciation that cancer patients who have a lower overall survival rate when treated with immune checkpoint pathway inhibitors, such as a CTLA-4 inhibitor, tend to have a larger number of clonotypes that undergo a reduction in frequency after or during such treatment.

In some embodiments, each of the first and second clonotype profiles comprise at least 10³ clonotypes; or at least 10⁴ clonotypes; or at least 10⁵ clonotypes; or at least 10⁶ clonotypes. In some embodiments, a baseline set of clonotypes and their frequencies is determined from a first clonotype profile of a patient. Such a baseline set may comprise all clonotypes having a frequency greater than 10⁻⁶; or such a baseline set may comprise all clonotypes having a frequency greater than 10⁻⁵; or such a baseline set may comprise all clonotypes having a frequency greater than 10⁻⁴. In such embodiments, frequencies of clonotypes of the baseline set are compared to frequencies of the same clonotypes in one or more second clonotype profiles. In some embodiments, the predetermined value is in a range of from 10 to 1000 clonotypes; that is, in some embodiments of the method, a prediction of lack of responsiveness is indicated whenever frequencies of a number of clonotypes (in the range of from 10 to 1000) from the baseline set decrease in a second clonotype profile of the patient. In other embodiments, the predetermined value is a fraction of clonotype frequencies measured. In some embodiments, a predetermined value of clonotypes that decrease in frequency in a successive clonotype profile is at least 10 percent of clonotype frequencies measured in the first clonotype profile, or at least 10 percent of clonotype frequencies of clonotypes in a baseline set. In still other embodiments, the predetermined value of clonotypes that decrease in frequency in a successive clonotype profile is at least 20 percent of clonotype frequencies measured in the first clonotype profile, or at least 20 percent of clonotype frequencies of clonotypes in a baseline set. In still other embodiments, at least the 100 highest frequency clonotypes of a first clonotype profile are compared to frequencies of the same clonotypes in a second clonotype profile. In other embodiments, at least the 1000 highest frequency clonotypes of a first clonotype profile are compared to the frequencies of the same clonotypes a second clonotype profile. In some embodiment, frequency changes are only determined for clonotypes of the first clonotype profile which are present with a frequency of greater than 10⁻⁶, or greater than 10⁻⁵, or greater than 10⁻⁴, or greater than 10⁻³. In some embodiments, frequencies of the 100 highest frequency clonotypes of a first clonotype profile are compared with the frequencies of the same clonotypes in a second clonotype profile. In some embodiments, frequencies of the 1000 highest frequency clonotypes of a first clonotype profile are compared with the frequencies of the same clonotypes in a second clonotype profile. In some embodiments, frequency changes are determined and counted whenever such changes are statistically significant. In some embodiments, frequency changes are determined and counted whenever such changes are at least a two-fold decrease (i.e. a reduction by a factor of ½); or a five-fold decrease (i.e. a reduction by a factor of 0.2). In some embodiments, immune checkpoint pathway inhibitors are monoclonal antibodies or antigen-binding fragments thereof which are specific for proteins in such pathways. In particular, such CTLA-4 inhibitors include ipilimumab and tremelimumab and such PD-1 inhibitors include nivolumab.

The size and type of clonotype profiles used with the method may vary widely in various embodiments of the invention. In some embodiments, clonotype profiles comprise at least 10³ clonotypes; in other embodiments, clonotype profiles comprise at least 10⁴ clonotypes; in still other embodiments, clonotype profiles comprise at least 10⁵ clonotypes. In some embodiments, rearranged nucleic acids of clonotypes may be 25-200 nucleotide segments of a VDJ rearrangement of TCR β, a DJ rearrangement of TCR β, a VJ rearrangement of TCR α, a VJ rearrangement of TCR γ, a VDJ rearrangement of TCR δ, a VD rearrangement of TCR δ, or the like. In another embodiment, rearranged nucleic acids of clonotypes may be 25-200 nucleotide segments of a VDJ rearrangement of TCR β. Techniques for generating clonotype profiles are well-known and are described in the following references, which are incorporated by reference: Faham and Willis, U.S. Pat. Nos. 8,236,503; 8,748,103 and 8,691,503; and U.S. patent publications 2013/0236895; 2010/0021896; and the like.

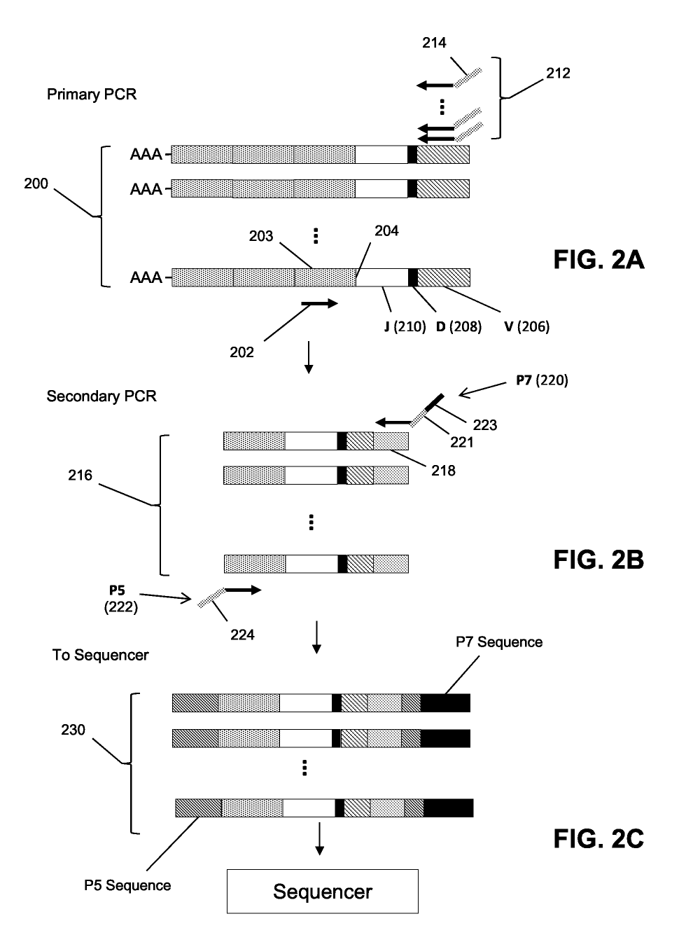

One embodiment for generating a clonotype profile of nucleic acids encoding TCRβ chains is illustrated in FIGS. 2A and 2B where RNA encoding TCRβ is amplified in a two-staged PCR. As described more fully below, primer (202) and primer set (212) are used in a first stage amplification to attach common primer binding site (214) to all the nucleic acids encoding TCRβs. FIG. 2B illustrates the components of a second stage amplification for generating more material and for attaching primer binding sites P5 (222) and P7 (220) which are used in cluster formation (via bridge PCR) in the Solexa-based sequencing protocol. Primer P7 (220) may also include sample tag (221) for multiplexing up to 96 samples for concurrent sequencing in the same run, e.g. Illumina application note 770-2008-011 (2008). A different type of tag in the same primer may be used to increase the accuracy of the determination of receptor chain frequencies. In this embodiment, primer P7 is modified to include a highly diverse tag set, so that instead of 96 tags, primer P7 is engineered to have 10,000 distinct tags, or more. In other words, primer P7 is a mixture of 10,000 or more distinct oligonucleotides each having an identical template binding region, a distinct tag sequence, and an identical 5′ tail portion (e.g., (223) in FIG. 2B). With this arrangement, any subset of nucleic acids encoding the same receptor chain (e.g. less than 100) will receive a different tag with high probability. Such a process of pairing members of a small set of nucleic acids with a much larger set of tags for counting, labeling, sorting purposes is well known and is disclosed in various forms in the following references that are incorporated by reference, Brenner, U.S. Pat. No. 6,172,214; Brenner et al, U.S. Pat. No. 7,537,897; and Macevicz, International patent publication WO US2005/111242; Brenner et al, Proc. Natl. Acad. Sci., 97: 1665-1670 (2000); Casbon et al, Nucleic Acids Research, 39(12): e81 (2011); Fu et al, Proc. Natl. Acad. Sci., 108: 9026-9031 (2011). Construction of sets of minimally cross-hybridizing oligonucleotide tag, or tags with other useful properties, is disclosed in the following exemplary references, which are incorporated by reference: Brenner, U.S. Pat. No. 6,172,214; Morris et al, U.S. patent publication 2004/0146901; Mao et al, U.S. patent publication 2005/0260570; and the like. Preferably, the tag set should be at least 100 times (or more) the size of the set of nucleic acids to be labeled if all nucleic acids are to receive a unique tag with high probability. For immune receptor chains, in one embodiment, the number of distinct tags is in the range of from 10,000 to 100,000; in another embodiment, the number of distinct tags is in the range of from 10,000 to 50,000; and in another embodiment, the number of distinct tags is in the range of from 10,000 to 20,000. As disclosed in Brenner, U.S. Pat. No. 6,172,214, such large mixtures of oligonucleotide tags may be synthesized by combinatorial methods; alternatively, primers containing unique tags may be synthesized individually by non-combinatorial methods, such as disclosed by Cleary et al, Nature Methods, 1: 241-248 (2004); York et al, Nucleic Acids Research, 40(1): e4 (2012); LeProust et al, Nucleic Acids Research, 38(8): 2522-2540 (2010); and the like.

As described more fully below, in some embodiments, clonotype profiles for use in the invention may be generated using the following steps: (a) obtaining a sample of nucleic acids from T-cells and/or cell free DNA or RNA of an individual; (b) amplifying from the sample in a multiplex polymerase chain reaction (PCR) recombined nucleic acids comprising complementary determining region 3 (CDR3) sequences from T cell receptor genes; (c) spatially isolating individual molecules of the amplified recombined nucleic acids; (d) sequencing by synthesis using reversibly terminated labeled nucleotides the spatially isolated recombined nucleic acids to generate at least 10,000 sequence reads each having at least 30 bp; (e) coalescing the sequence reads into clonotypes of the recombined nucleic acids, wherein sequence reads are coalesced into different clonotypes whenever said sequence reads are distinct with a confidence of at least 99 percent; and (f) quantifying clonotypes by counting sequence reads thereof. In other embodiments, clonotype profiles for use in the invention may be generated using the following steps: (a) obtaining a sample from the individual comprising T-cells and/or cell-free DNA or RNA; (b) amplifying from the sample in a multiplex polymerase chain reaction (PCR) molecules of recombined nucleic acid comprising complementary determining region 3 (CDR3) sequences from T-cell receptor genes; (c) spatially isolating individual molecules of the amplified recombined nucleic acids; (d) sequencing by synthesis the spatially isolated recombined nucleic acids to provide sequence reads of CDR3 sequences, wherein said sequencing includes incorporating by a polymerase one or more nucleoside triphosphates at the end of a sequencing primer hybridized to said recombined nucleic acids and detection thereof by a change in current; (e) coalescing the sequence reads into clonotypes of the recombined nucleic acids, wherein sequence reads are coalesced into different clonotypes whenever said sequence reads are distinct with a confidence of at least 99 percent; and (f) quantifying clonotypes by counting sequence reads thereof.

In one aspect, methods of the invention may be used with treatment of solid tumors, such as melanoma, prostate cancer, or the like. In another aspect, methods of the invention may be used with treatment of lymphoid and myeloid proliferative disorders. In another aspect, methods of the invention are applicable to lymphomas and leukemias. In another aspect, methods of the invention are applicable lymphomas or leukemias, such as follicular lymphoma, chronic lymphocytic leukemia (CLL), acute lymphocytic leukemia (ALL), chronic myelogenous leukemia (CML), acute myelogenous leukemia (AML), Hodgkins's and non-Hodgkin's lymphomas, multiple myeloma (MM), monoclonal gammopathy of undetermined significance (MGUS), mantle cell lymphoma (MCL), diffuse large B cell lymphoma (DLBCL), myelodysplastic syndromes (MDS), T cell lymphoma, or the like.

### Samples

Clonotype profiles may be obtained from samples of immune cells. For example, immune cells can include T-cells and/or B-cells. T-cells (T lymphocytes) include, for example, cells that express T cell receptors. T-cells include helper T cells (effector T cells or Th cells), cytotoxic T cells (CTLs), memory T cells, and regulatory T cells. In one aspect a sample of T cells includes at least 1,000 T cells; but more typically, a sample includes at least 10,000 T cells, and more typically, at least 100,000 T cells. In another aspect, a sample includes a number of T cells in the range of from 1000 to 1,000,000 cells.

Samples used in the methods of the invention can come from a variety of tissues, including, for example, tumor tissue, blood and blood plasma, lymph fluid, cerebrospinal fluid surrounding the brain and the spinal cord, synovial fluid surrounding bone joints, and the like. In one embodiment, the sample is a blood sample. The blood sample can be about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mL. The sample can be a tumor biopsy. The biopsy can be from, for example, from a tumor of the brain, liver, lung, heart, colon, kidney, or bone marrow. Any biopsy technique used by those skilled in the art can be used for isolating a sample from a subject. For example, a biopsy can be an open biopsy, in which general anesthesia is used. The biopsy can be a closed biopsy, in which a smaller cut is made than in an open biopsy. The biopsy can be a core or incisional biopsy, in which part of the tissue is removed. The biopsy can be an excisional biopsy, in which attempts to remove an entire lesion are made. The biopsy can be a fine needle aspiration biopsy, in which a sample of tissue or fluid is removed with a needle.

A sample for use with the invention can include DNA (e.g., genomic DNA) or RNA (e.g., messenger RNA). The nucleic acid can be cell-free DNA or RNA, e.g. extracted from the circulatory system, Vlassov et al, Curr. Mol. Med., 10: 142-165 (2010); Swarup et al, FEBS Lett., 581: 795-799 (2007). In the methods of the provided invention, the amount of RNA or DNA from a subject that can be analyzed includes, for example, as low as a single cell in some applications (e.g., a calibration test with other cell selection criteria, e.g. morphological criteria) and as many as 10 million of cells or more, which translates into a quantity of DNA in the range of from 6 pg-60 ug, and a quantity of RNA in the range of from 1 pg-10 ug. In some embodiments, a nucleic acid sample is a DNA sample of from 6 pg to 60 ug. In other embodiments, a nucleic acid sample is a DNA sample from 100 L to 10 mL of peripheral blood; in other embodiments, a nucleic acid sample is a DNA sample from a cell free fraction of from 100 μL to 10 mL of peripheral blood.

In some embodiments, a sample of lymphocytes or cell free nucleic acid is sufficiently large so that substantially every T cell with a distinct clonotype is represented therein, thereby forming a “repertoire” of clonotypes. In one embodiment, to achieve substantial representation of every distinct clonotype, a sample is taken that contains with a probability of ninety-nine percent every clonotype of a population present at a frequency of 0.01 percent or greater. In another embodiment, a sample is taken that contains with a probability of ninety-nine percent every clonotype of a population present at a frequency of 0.001 percent or greater. In another embodiment, a sample is taken that contains with a probability of ninety-nine percent every clonotype of a population present at a frequency of 0.0001 percent or greater. And in another embodiment, a sample is taken that contains with a probability of ninety-nine percent every clonotype of a population present at a frequency of 0.00001 percent or greater. In another embodiment, a sample is taken that contains with a probability of ninety-five percent every clonotype of a population present at a frequency of 0.001 percent or greater. In one embodiment, a sample of T cells includes at least one half million cells, and in another embodiment such sample includes at least one million T cells.

Nucleic acid samples may be obtained from peripheral blood using conventional techniques, e.g. Innis et al, editors, PCR Protocols (Academic Press, 1990); or the like. For example, white blood cells may be separated from blood samples using convention techniques, e.g. RosetteSep kit (Stem Cell Technologies, Vancouver, Canada). Blood samples may range in volume from 100 μL to 10 mL; in one aspect, blood sample volumes are in the range of from 100 μL to 2 mL. DNA and/or RNA may then be extracted from such blood sample using conventional techniques for use in methods of the invention, e.g. DNeasy Blood & Tissue Kit (Qiagen, Valencia, Calif.). Optionally, subsets of white blood cells, e.g. lymphocytes, may be further isolated using conventional techniques, e.g. fluorescently activated cell sorting (FACS)(Becton Dickinson, San Jose, Calif.), magnetically activated cell sorting (MACS)(Miltenyi Biotec, Auburn, Calif.), or the like

Cell-free DNA may also be extracted from peripheral blood samples using conventional techniques, e.g. Lo et al, U.S. Pat. No. 6,258,540; Huang et al, Methods Mol. Biol., 444: 203-208 (2008); and the like, which are incorporated herein by reference. By way of nonlimiting example, peripheral blood may be collected in EDTA tubes, after which it may be fractionated into plasma, white blood cell, and red blood cell components by centrifugation. DNA from the cell free plasma fraction (e.g. from 0.5 to 2.0 mL) may be extracted using a QIAamp DNA Blood Mini Kit (Qiagen, Valencia, Calif.), or like kit, in accordance with the manufacturer's protocol.

Whenever a source of material from which a sample is taken is scarce, such as, clinical study samples, or the like, DNA from the material may be amplified by a non-biasing technique, such as whole genome amplification (WGA), multiple displacement amplification (MDA); or like technique, e.g. Hawkins et al, Curr. Opin. Biotech., 13: 65-67 (2002); Dean et al, Genome Research, 11: 1095-1099 (2001); Wang et al, Nucleic Acids Research, 32: e76 (2004); Hosono et al, Genome Research, 13: 954-964 (2003); and the like.

Since the identifying recombinations are present in the DNA of each individual's adaptive immunity cells as well as their associated RNA transcripts, either RNA or DNA can be sequenced in the methods of the provided invention. A recombined sequence from a T-cell encoding a T cell receptor molecule, or a portion thereof, is referred to as a clonotype. The DNA and RNA can correspond to sequences encoding α, β, γ, or δ chains of a TCR. In a majority of T-cells, the TCR is a heterodimer consisting of an α-chain and β-chain. The TCRα chain is generated by VJ recombination, and the β chain receptor is generated by V(D)J recombination. For the TCRβ chain, in humans there are 48 V segments, 2 D segments, and 13 J segments. Several bases may be deleted and others added (called N and P nucleotides) at each of the two junctions. In a minority of T-cells, the TCRs consist of γ and δ delta chains. The TCR γ chain is generated by VJ recombination, and the TCR 8 chain is generated by V(D)J recombination (Kenneth Murphy, Paul Travers, and Mark Walport, Janeway's Immunology 7th edition, Garland Science, 2007, which is herein incorporated by reference in its entirety).

Adequate sampling of the cells is an important aspect of interpreting the repertoire data, as described further below in the definitions of “clonotype” and “repertoire.” For example, starting with 1,000 cells creates a minimum frequency that the assay is sensitive to regardless of how many sequencing reads are obtained. Therefore one aspect of this invention is the development of methods to quantitate the number of input immune receptor molecules. This has been implemented this for TCRβ and IgH sequences. In either case the same set of primers are used that are capable of amplifying all the different sequences. In order to obtain an absolute number of copies, a real time PCR with the multiplex of primers is performed along with a standard with a known number of immune receptor copies. This real time PCR measurement can be made from the amplification reaction that will subsequently be sequenced or can be done on a separate aliquot of the same sample. In the case of DNA, the absolute number of rearranged immune receptor molecules can be readily converted to number of cells (within 2 fold as some cells will have 2 rearranged copies of the specific immune receptor assessed and others will have one). In the case of cDNA the measured total number of rearranged molecules in the real time sample can be extrapolated to define the total number of these molecules used in another amplification reaction of the same sample. In addition, this method can be combined with a method to determine the total amount of RNA to define the number of rearranged immune receptor molecules in a unit amount (say 1 μg) of RNA assuming a specific efficiency of cDNA synthesis. If the total amount of cDNA is measured then the efficiency of cDNA synthesis need not be considered. If the number of cells is also known then the rearranged immune receptor copies per cell can be computed. If the number of cells is not known, one can estimate it from the total RNA as cells of specific type usually generate comparable amount of RNA. Therefore from the copies of rearranged immune receptor molecules per 1 μg one can estimate the number of these molecules per cell.

An approach that can be utilized to determine absolute numbers of clonotypes in a sample is to add a known amount of unique immune receptor rearranged molecules with a known sequence, i.e. known amounts of one or more internal standards, to the cDNA or genomic DNA from a sample of unknown quantity. By counting the relative number of molecules that are obtained for the known added sequence compared to the rest of the sequences of the same sample, one can estimate the number of rearranged immune receptor molecules in the initial cDNA sample. (Such techniques for molecular counting are well-known, e.g. Brenner et al, U.S. Pat. No. 7,537,897, which is incorporated herein by reference). Data from sequencing the added unique sequence can be used to distinguish the different possibilities if a real time PCR calibration is being used as well. Low copy number of rearranged immune receptor in the DNA (or cDNA) would create a high ratio between the number of molecules for the spiked sequence compared to the rest of the sample sequences. On the other hand, if the measured low copy number by real time PCR is due to inefficiency in the reaction, the ratio would not be high.

### Amplification of Nucleic Acid Populations

Amplicons of target populations of nucleic acids may be generated by a variety of amplification techniques. In one aspect of the invention, multiplex PCR is used to amplify members of a mixture of nucleic acids, particularly mixtures comprising recombined immune molecules such as T cell receptors, or portions thereof. Guidance for carrying out multiplex PCRs of such immune molecules is found in the following references, which are incorporated by reference: Morley, U.S. Pat. No. 5,296,351; Gorski, U.S. Pat. No. 5,837,447; Dau, U.S. Pat. No. 6,087,096; Von Dongen et al, U.S. patent publication 2006/0234234; European patent publication EP 1544308B1; and the like.

After amplification of DNA from the genome (or amplification of nucleic acid in the form of cDNA by reverse transcribing RNA), the individual nucleic acid molecules can be isolated, optionally re-amplified, and then sequenced individually. Exemplary amplification protocols may be found in van Dongen et al, Leukemia, 17: 2257-2317 (2003) or van Dongen et al, U.S. patent publication 2006/0234234, which is incorporated by reference. Briefly, an exemplary protocol is as follows: Reaction buffer: ABI Buffer II or ABI Gold Buffer (Life Technologies, San Diego, Calif.); 50 μL final reaction volume; 100 ng sample DNA; 10 pmol of each primer (subject to adjustments to balance amplification as described below); dNTPs at 200 μM final concentration; MgCl₂ at 1.5 mM final concentration (subject to optimization depending on target sequences and polymerase); Taq polymerase (1-2 U/tube); cycling conditions: preactivation 7 min at 95° C.; annealing at 60° C.; cycling times: 30 s denaturation; 30 s annealing; 30 s extension. Polymerases that can be used for amplification in the methods of the invention are commercially available and include, for example, Taq polymerase, AccuPrime polymerase, or Pfu. The choice of polymerase to use can be based on whether fidelity or efficiency is preferred.

Real time PCR, picogreen staining, nanofluidic electrophoresis (e.g. LabChip) or UV absorption measurements can be used in an initial step to judge the functional amount of amplifiable material.

In one aspect, multiplex amplifications are carried out so that relative amounts of sequences in a starting population are substantially the same as those in the amplified population, or amplicon. That is, multiplex amplifications are carried out with minimal amplification bias among member sequences of a sample population. In one embodiment, such relative amounts are substantially the same if each relative amount in an amplicon is within five fold of its value in the starting sample. In another embodiment, such relative amounts are substantially the same if each relative amount in an amplicon is within two fold of its value in the starting sample. As discussed more fully below, amplification bias in PCR may be detected and corrected using conventional techniques so that a set of PCR primers may be selected for a predetermined repertoire that provide unbiased amplification of any sample.

In regard to many repertoires based on TCR or BCR sequences, a multiplex amplification optionally uses all the V segments. The reaction is optimized to attempt to get amplification that maintains the relative abundance of the sequences amplified by different V segment primers. Some of the primers are related, and hence many of the primers may “cross talk,” amplifying templates that are not perfectly matched with it. The conditions are optimized so that each template can be amplified in a similar fashion irrespective of which primer amplified it. In other words if there are two templates, then after 1,000 fold amplification both templates can be amplified approximately 1,000 fold, and it does not matter that for one of the templates half of the amplified products carried a different primer because of the cross talk. In subsequent analysis of the sequencing data the primer sequence is eliminated from the analysis, and hence it does not matter what primer is used in the amplification as long as the templates are amplified equally.

In one embodiment, amplification bias may be avoided by carrying out a two-stage amplification (as described in Faham and Willis, cited above) wherein a small number of amplification cycles are implemented in a first, or primary, stage using primers having tails non-complementary with the target sequences. The tails include primer binding sites that are added to the ends of the sequences of the primary amplicon so that such sites are used in a second stage amplification using only a single forward primer and a single reverse primer, thereby eliminating a primary cause of amplification bias. Preferably, the primary PCR will have a small enough number of cycles (e.g. 5-10) to minimize the differential amplification by the different primers. The secondary amplification is done with one pair of primers and hence the issue of differential amplification is minimal. One percent of the primary PCR is taken directly to the secondary PCR. Thirty-five cycles (equivalent to ˜28 cycles without the 100 fold dilution step) used between the two amplifications were sufficient to show a robust amplification irrespective of whether the breakdown of cycles were: one cycle primary and 34 secondary or 25 primary and 10 secondary. Even though ideally doing only 1 cycle in the primary PCR may decrease the amplification bias, there are other considerations. One aspect of this is representation. This plays a role when the starting input amount is not in excess to the number of reads ultimately obtained. For example, if 1,000,000 reads are obtained and starting with 1,000,000 input molecules then taking only representation from 100,000 molecules to the secondary amplification would degrade the precision of estimating the relative abundance of the different species in the original sample. The 100 fold dilution between the 2 steps means that the representation is reduced unless the primary PCR amplification generated significantly more than 100 molecules. This indicates that a minimum 8 cycles (256 fold), but more comfortably 10 cycle (˜1,000 fold), may be used. The alternative to that is to take more than 1% of the primary PCR into the secondary but because of the high concentration of primer used in the primary PCR, a big dilution factor is can be used to ensure these primers do not interfere in the amplification and worsen the amplification bias between sequences. Another alternative is to add a purification or enzymatic step to eliminate the primers from the primary PCR to allow a smaller dilution of it. In this example, the primary PCR was 10 cycles and the second 25 cycles.

### Generating Sequence Reads for Clonotypes

Any high-throughput technique for sequencing nucleic acids can be used in the method of the invention. Preferably, such technique has a capability of generating in a cost-effective manner a volume of sequence data from which at least 1000 clonotypes can be determined, and preferably, from which at least 10,000 to 1,000,000 clonotypes can be determined. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches can thus be used to study the repertoire of T-cell receptor (TCR) and/or B-cell receptor (BCR). In one aspect of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm².

In one aspect, a sequence-based clonotype profile of an individual is obtained using the following steps: (a) obtaining a nucleic acid sample from T-cells of the individual; (b) spatially isolating individual molecules derived from such nucleic acid sample, the individual molecules comprising at least one template generated from a nucleic acid in the sample, which template comprises a somatically rearranged region or a portion thereof, each individual molecule being capable of producing at least one sequence read; (c) sequencing said spatially isolated individual molecules; and (d) determining abundances of different sequences of the nucleic acid molecules from the nucleic acid sample to generate the clonotype profile. In one embodiment, each of the somatically rearranged regions comprise a V region and a J region. In another embodiment, the step of sequencing comprises bidirectionally sequencing each of the spatially isolated individual molecules to produce at least one forward sequence read and at least one reverse sequence read. Further to the latter embodiment, at least one of the forward sequence reads and at least one of the reverse sequence reads have an overlap region such that bases of such overlap region are determined by a reverse complementary relationship between such sequence reads. In still another embodiment, each of the somatically rearranged regions comprise a V region and a J region and the step of sequencing further includes determining a sequence of each of the individual nucleic acid molecules from one or more of its forward sequence reads and at least one reverse sequence read starting from a position in a J region and extending in the direction of its associated V region. In another embodiment, individual molecules comprise nucleic acids selected from the group consisting of TCRβ molecules, TCRγ molecules, complete TCRδ molecules, and incomplete TCRδ molecules.

In one aspect, for each sample from an individual, the sequencing technique used in the methods of the invention generates sequences of least 1000 clonotypes per run; in another aspect, such technique generates sequences of at least 10,000 clonotypes per run; in another aspect, such technique generates sequences of at least 100,000 clonotypes per run; in another aspect, such technique generates sequences of at least 500,000 clonotypes per run; and in another aspect, such technique generates sequences of at least 1,000,000 clonotypes per run. In still another aspect, such technique generates sequences of between 100,000 to 1,000,000 clonotypes per run per individual sample. In some embodiments, each of the foregoing numbers of clonotypes is determined from at least 10 sequence reads.

The sequencing technique used in the methods of the provided invention can generate about 30 bp, about 40 bp, about 50 bp, about 60 bp, about 70 bp, about 80 bp, about 90 bp, about 100 bp, about 110, about 120 bp per read, about 150 bp, about 200 bp, about 250 bp, about 300 bp, about 350 bp, about 400 bp, about 450 bp, about 500 bp, about 550 bp, or about 600 bp per read.

### Clonotype Determination from Sequence Data

Constructing clonotypes from sequence read data depends in part on the sequencing method used to generate such data, as the different methods have different expected read lengths and data quality. In one approach, a Solexa sequencer is employed to generate sequence read data for analysis. In one embodiment, a sample is obtained that provides at least 0.5-1.0×10⁶ lymphocytes to produce at least 1 million template molecules, which after optional amplification may produce a corresponding one million or more clonal populations of template molecules (or clusters). For most high throughput sequencing approaches, including the Solexa approach, such over sampling at the cluster level is desirable so that each template sequence is determined with a large degree of redundancy to increase the accuracy of sequence determination. For Solexa-based implementations, preferably the sequence of each independent template is determined 10 times or more. For other sequencing approaches with different expected read lengths and data quality, different levels of redundancy may be used for comparable accuracy of sequence determination. Those of ordinary skill in the art recognize that the above parameters, e.g. sample size, redundancy, and the like, are design choices related to particular applications.

In one aspect of the invention, sequences of clonotypes (including but not limited to those derived from TCRα, TCRβ, TCRγ, or TCRδ) may be determined by combining information from one or more sequence reads, for example, along the V(D)J regions of the selected chains. In another aspect, sequences of clonotypes are determined by combining information from a plurality of sequence reads. Such pluralities of sequence reads may include one or more sequence reads along a sense strand (i.e. “forward” sequence reads) and one or more sequence reads along its complementary strand (i.e. “reverse” sequence reads). When multiple sequence reads are generated along the same strand, separate templates are first generated by amplifying sample molecules with primers selected for the different positions of the sequence reads.

Sequence reads of the invention may have a wide variety of lengths, depending in part on the sequencing technique being employed. For example, for some techniques, several trade-offs may arise in its implementation, for example, (i) the number and lengths of sequence reads per template and (ii) the cost and duration of a sequencing operation. In one embodiment, sequence reads are in the range of from 20 to 400 nucleotides; in another embodiment, sequence reads are in a range of from 30 to 200 nucleotides; in still another embodiment, sequence reads are in the range of from 30 to 120 nucleotides. In one embodiment, 1 to 4 sequence reads are generated for determining the sequence of each clonotype; in another embodiment, 2 to 4 sequence reads are generated for determining the sequence of each clonotype; and in another embodiment, 2 to 3 sequence reads are generated for determining the sequence of each clonotype. In the foregoing embodiments, the numbers given are exclusive of sequence reads used to identify samples from different individuals.

In another aspect of the invention, sequences of clonotypes are determined in part by aligning sequence reads to one or more V region reference sequences and one or more J region reference sequences, and in part by base determination without alignment to reference sequences, such as in the highly variable NDN region. A variety of alignment algorithms may be applied to the sequence reads and reference sequences. For example, guidance for selecting alignment methods is available in Batzoglou, Briefings in Bioinformatics, 6: 6-22 (2005), which is incorporated by reference. In one aspect, whenever V reads or C reads (as mentioned above) are aligned to V and J region reference sequences, a tree search algorithm is employed, e.g. as described generally in Gusfield (cited above) and Cormen et al, Introduction to Algorithms, Third Edition (The MIT Press, 2009).

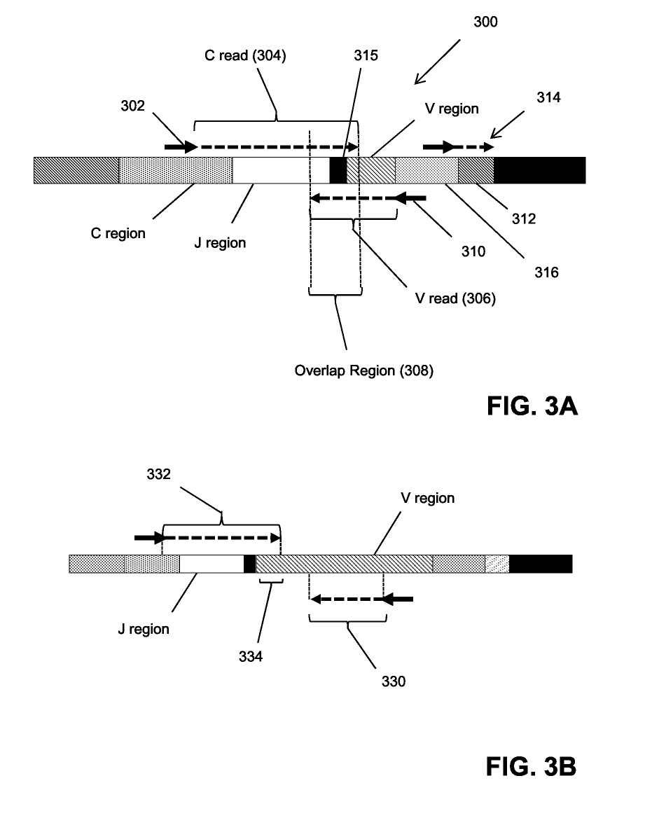

In another aspect, an end of at least one forward read and an end of at least one reverse read overlap in an overlap region (e.g. 308 in FIG. 3B), so that the bases of the reads are in a reverse complementary relationship with one another. Thus, for example, if a forward read in the overlap region is “5′-acgttgc”, then a reverse read in a reverse complementary relationship is “5′-gcaacgt” within the same overlap region. In one aspect, bases within such an overlap region are determined, at least in part, from such a reverse complementary relationship. That is, a likelihood of a base call (or a related quality score) in a prospective overlap region is increased if it preserves, or is consistent with, a reverse complementary relationship between the two sequence reads. In one aspect, clonotypes of TCR β and IgH chains (illustrated in FIG. 3B) are determined by at least one sequence read starting in its J region and extending in the direction of its associated V region (referred to herein as a “C read” (304)) and at least one sequence read starting in its V region and extending in the direction of its associated J region (referred to herein as a “V read” (306)). Overlap region (308) may or may not encompass the NDN region (315) as shown in FIG. 3B. Overlap region (308) may be entirely in the J region, entirely in the NDN region, entirely in the V region, or it may encompass a J region-NDN region boundary or a V region-NDN region boundary, or both such boundaries (as illustrated in FIG. 3B). Typically, such sequence reads are generated by extending sequencing primers, e.g. (302) and (310) in FIG. 3B, with a polymerase in a sequencing-by-synthesis reaction, e.g. Metzger, Nature Reviews Genetics, 11: 31-46 (2010); Fuller et al, Nature Biotechnology, 27: 1013-1023 (2009). The binding sites for primers (302) and (310) are predetermined, so that they can provide a starting point or anchoring point for initial alignment and analysis of the sequence reads. In one embodiment, a C read is positioned so that it encompasses the D and/or NDN region of the TCR β chain and includes a portion of the adjacent V region, e.g. as illustrated in FIGS. 3B and 3C. In one aspect, the overlap of the V read and the C read in the V region is used to align the reads with one another. In other embodiments, such alignment of sequence reads is not necessary, e.g. with TCRβ chains, so that a V read may only be long enough to identify the particular V region of a clonotype. This latter aspect is illustrated in FIG. 3C. Sequence read (330) is used to identify a V region, with or without overlapping another sequence read, and another sequence read (332) traverses the NDN region and is used to determine the sequence thereof. Portion (334) of sequence read (332) that extends into the V region is used to associate the sequence information of sequence read (332) with that of sequence read (330) to determine a clonotype. For some sequencing methods, such as base-by-base approaches like the Solexa sequencing method, sequencing run time and reagent costs are reduced by minimizing the number of sequencing cycles in an analysis. Optionally, as illustrated in FIG. 3B, amplicon (300) is produced with sample tag (312) to distinguish between clonotypes originating from different biological samples, e.g. different patients. Sample tag (312) may be identified by annealing a primer to primer binding region (316) and extending it (314) to produce a sequence read across tag (312), from which sample tag (312) is decoded.

### TCRβ Repertoire Analysis

In this example, TCRβ chains are analyzed. The analysis includes amplification, sequencing, and analyzing the TCRβ sequences. One primer is complementary to a common sequence in Cβ1 and Cβ2, and there are 34 V primers capable of amplifying all 48 V segments. Cβ1 or Cβ2 differ from each other at position 10 and 14 from the J/C junction. The primer for Cβ1 and Cβ2 ends at position 16 bp and has no preference for Cβ1 or Cβ2. The 34 V primers are modified from an original set of primers disclosed in Van Dongen et al, U.S. patent publication 2006/0234234, which is incorporated herein by reference. The modified primers are disclosed in Faham et al, U.S. patent publication 2010/0151471, which is also incorporated herein by reference.

The Illumina Genome Analyzer is used to sequence the amplicon produced by the above primers. A two-stage amplification is performed on messenger RNA transcripts (200), as illustrated in FIGS. 2A-2B, the first stage employing the above primers and a second stage to add common primers for bridge amplification and sequencing. As shown in FIG. 2A, a primary PCR is performed using on one side a 20 bp primer (202) whose 3′ end is 16 bases from the J/C junction (204) and which is perfectly complementary to Cβ1(203) and the two alleles of Cβ2. In the V region (206) of RNA transcripts (200), primer set (212) is provided which contains primer sequences complementary to the different V region sequences (34 in one embodiment). Primers of set (212) also contain a non-complementary tail (214) that produces amplicon (216) having primer binding site (218) specific for P7 primers (220). After a conventional multiplex PCR, amplicon (216) is formed that contains the highly diverse portion of the J(D)V region (206, 208, and 210) of the mRNA transcripts and common primer binding sites (203 and 218) for a secondary amplification to add a sample tag (221) and primers (220 and 222) for cluster formation by bridge PCR. In the secondary PCR, on the same side of the template, a primer (222 in FIG. 2B and referred to herein as “C10-17-P5”) is used that has at its 3′ end the sequence of the 10 bases closest to the J/C junction, followed by 17 bp with the sequence of positions 15-31 from the J/C junction, followed by the P5 sequence (224), which plays a role in cluster formation by bridge PCR in Solexa sequencing. (When the C10-17-P5 primer (222) anneals to the template generated from the first PCR, a 4 bp loop (position 11-14) is created in the template, as the primer hybridizes to the sequence of the 10 bases closest to the J/C junction and bases at positions 15-31 from the J/C junction. The looping of positions 11-14 eliminates differential amplification of templates carrying Cβ1 or Cβ2. Sequencing is then done with a primer complementary to the sequence of the 10 bases closest to the J/C junction and bases at positions 15-31 from the J/C junction (this primer is called C′). C10-17-P5 primer can be HPLC purified in order to ensure that all the amplified material has intact ends that can be efficiently utilized in the cluster formation.)

In FIG. 2A, the length of the overhang on the V primers (212) is preferably 14 bp. The primary PCR is helped with a shorter overhang (214). Alternatively, for the sake of the secondary PCR, the overhang in the V primer is used in the primary PCR as long as possible because the secondary PCR is priming from this sequence. A minimum size of overhang (214) that supports an efficient secondary PCR was investigated. Two series of V primers (for two different V segments) with overhang sizes from 10 to 30 with 2 bp steps were made. Using the appropriate synthetic sequences, the first PCR was performed with each of the primers in the series and gel electrophoresis was performed to show that all amplified.

As illustrated in FIG. 2A, the primary PCR uses 34 different V primers (212) that anneal to V region (206) of RNA templates (200) and contain a common 14 bp overhang on the 5′ tail. The 14 bp is the partial sequence of one of the Illumina sequencing primers (termed the Read 2 primer). The secondary amplification primer (220) on the same side includes P7 sequence, a tag (221), and Read 2 primer sequence (223) (this primer is called Read2_tagX_P7). The P7 sequence is used for cluster formation. Read 2 primer and its complement are used for sequencing the V segment and the tag respectively. A set of 96 of these primers with tags numbered 1 through 96 are created (see below). These primers are HPLC purified in order to ensure that all the amplified material has intact ends that can be efficiently utilized in the cluster formation.

As mentioned above, the second stage primer, C-10-17-P5 (222, FIG. 2B) has interrupted homology to the template generated in the first stage PCR. The efficiency of amplification using this primer has been validated. An alternative primer to C-10-17-P5, termed CsegP5, has perfect homology to the first stage C primer and a 5′ tail carrying P5. The efficiency of using C-10-17-P5 and CsegP5 in amplifying first stage PCR templates was compared by performing real time PCR. In several replicates, it was found that PCR using the C-10-17-P5 primer had little or no difference in efficiency compared with PCR using the CsegP5 primer.

Amplicon (230) resulting from the 2-stage amplification illustrated in FIGS. 2A-2C has the structure typically used with the Illumina sequencer as shown in FIG. 2C. Two primers that anneal to the outmost part of the molecule, Illumina primers P5 and P7 are used for solid phase amplification of the molecule (cluster formation). Three sequence reads are done per molecule. The first read of 100 bp is done with the C′ primer, which has a melting temperature that is appropriate for the Illumina sequencing process. The second read is 6 bp long only and is solely for the purpose of identifying the sample tag. It is generated using a tag primer provided by the manufacturer (IIlumina). The final read is the Read 2 primer, also provided by the manufacturer (Illumina). Using this primer, a 100 bp read in the V segment is generated starting with the 1st PCR V primer sequence.

### EXAMPLE

### Immune Repertoire Response in Prostate and Melanoma Patients to Treatment with CTLA-4 Inhibitors

In this example, effects of CTLA-4 blockade on T cell clonotype diversity were assessed by comparing sequence-based clonotype profiles from successive samples of patient tissues. Specifically, effects were assessed on clonotype profiles from prostate and melanoma patients before and during or after treatment with CLTA-4 inhibitors. Peripheral blood mononuclear cells were obtained from patients prior to and during treatment with anti-CTLA-4 antibody. Such samples were obtained from (i) 25 patients with metastatic castration resistant prostate cancer treated with ipilimumab and GM-CSF, and (ii) 21 patients with metastatic melanoma treated with tremelimumab. Clonotype profiles of nucleic acids encoding a portion of the TCRβ chains encompassing its CDR3 region using the methodologies disclosed above (specifically, the Sequenta, Inc., LYMPHOSight™ platform). Clonotype profiles were analyzed by a variety of tools, including single value measures of change between profiles (e.g. Morisita index) and measures of differential clonotype abundance between profiles (e.g. DESeq analysis); Anders et al, Genome Biology, 11: R106 (2010); Legendre and Legendre, Numerical Ecology (Elsevier, 1998); Magurran, Measurement of Biological Diversity (Wiley-Blackwell, 2003); Wolda, Oecologia (Berl), 50: 296-302 (1981); Faham et al, International patent publication WO/2013/036459; each of which is incorporated herein by reference.

Study Design. PBMC were cryopreserved from 25 CRPC patients treated with anti-CTLA-4 (ipilimumab; Bristol-Myers Squibb) and GM-CSF (sargramostim; Sanofi) concurrently in a single-center phase 1/II clinical trial at UCSF (ClinicalTrials.gov identifier: NCT00064129) as previously described in Fong et al, Cancer Research, 69: 609-615 (2009). Patients were treated with up to 4 doses of ipilimumab ranging from 1.5 mg/kg to 10 mg/kg and GM-CSF at 250 μg/m2/day. Anti-CTLA-4 antibody was administered every 4 weeks with GM-CSF given daily on the first 2 weeks of these cycles. Patient characteristics from the phase I study were previously described (Fong et al, cited above). The 21 assessed melanoma patients were enrolled in a phase II clinical trial of single agent tremelimumab at 15 mg/kg administered every 3 months at UCLA (ClinicalTrials.gov identifier: NCT00471887) and were previously characterized. e.g. von Euw et al, J. Transl. Med., 7: 35 (2009). Samples from these patients were available at baseline and 1 month post-treatment. Patients were not restricted by HLA alleles. Informed consent was obtained for all investigations. PBMC from untreated controls were obtained from Cellular Technology Limited.

TCRβ Clonotype Profiles. The amplification and sequencing of TCRβ repertoire were carried out as described above and as described in Klinger et al (cited below). Briefly RNA was isolated from cells using AllPrep DNA/RNA mini and/or micro kits, according to manufacturer's instructions (Qiagen). RNA was reverse transcribed to cDNA using Vilo kits (Life Technologies). cDNA was amplified using locus specific primer sets for TCRβ. This amplification reaction reproducibly amplified all possible RNA transcripts found in the sample containing the rearranged TCRβ locus regardless of which variable (V) segment and which common constant (C) region allele each rearranged molecule possessed, while appending the necessary sequences for cluster formation and sample indexing. Ultimately, 115 bp were sequenced from the C side sufficient to sequence through the junctional sequence from C to V. In addition 95 bp was obtained from the V-to-C direction providing ample sequence to map the V segment accurately. Clonotypes were identified and enumerated as described above and in Klinger et al, PlosONE 8: e74231 (2013). Briefly, all reads are mapped to V and J segments. Identical sequences of successfully mapped reads were grouped in clonotypes. The frequency of each clonotype in a sample was determined by calculating the number of sequencing reads for each clonotype divided by the total number of passed sequencing reads in the sample.

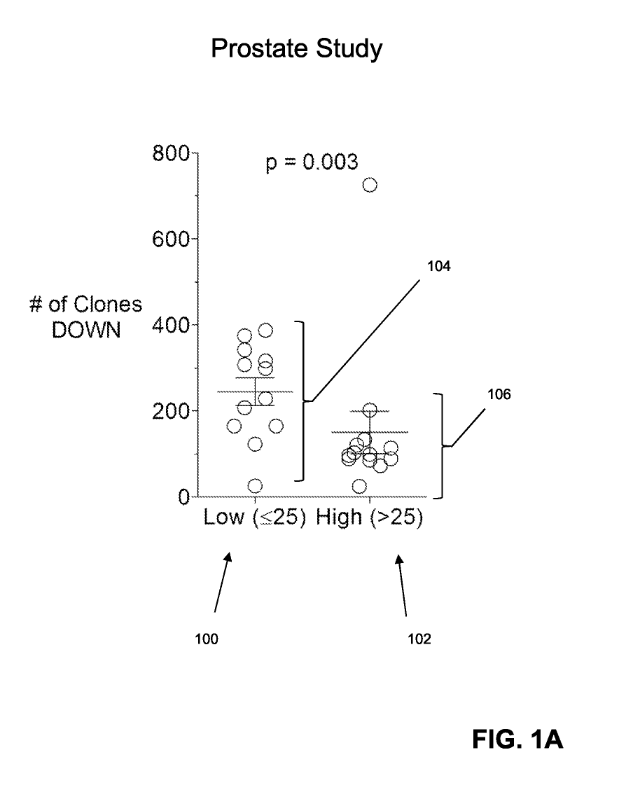

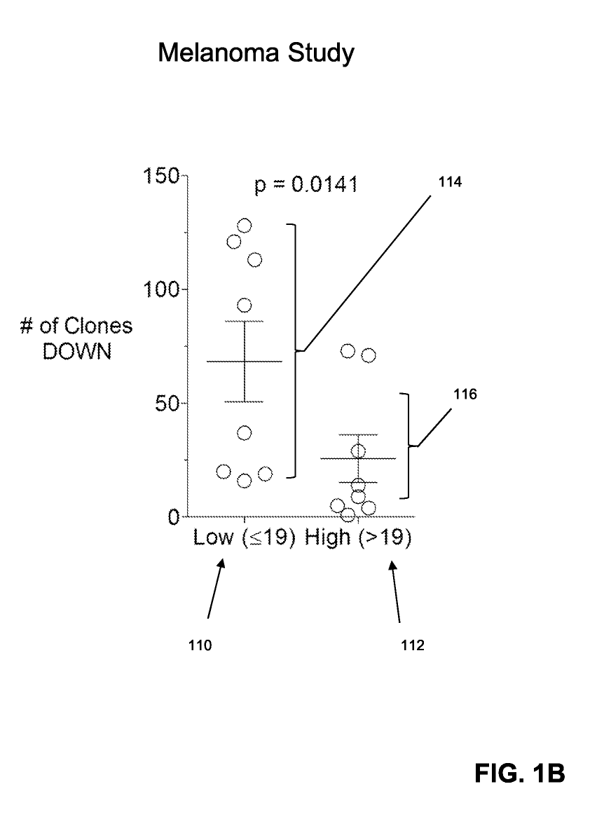

Data from the above study is shown in various figures discussed below. FIGS. 1A and 1B are based on the data from Tables I and II below. In FIG. 1A, prostate cancer patients have been divided into a group of low survivors (100) having overall survivals of less than 25 months and a group of high survivors (102) having overall survivals of more than 25 months. For each patient in each group the number of reduced frequency clonotypes (# of Clones DOWN) (measured at one month after initiation of treatment) was plotted. The data show that the high survivor group (on average) has a much lower number of reduced frequency clonotypes (106) and that the low survivor group (on average) has a higher number of reduced frequency clonotypes (104). In FIG. 1B, melanoma cancer patients have been divided into a group of low survivors (110) having overall survivals of less than 19 months and a group of high survivors (112) having overall survivals of more than 19 months. For each patient in each group the number of reduced frequency clonotypes (# of Clones DOWN) (measured at one month after initiation of treatment) was plotted. The data show that the high survivor group (on average) has a much lower number of reduced frequency clonotypes (116) and that the low survivor group (on average) has a higher number of reduced frequency clonotypes (114).

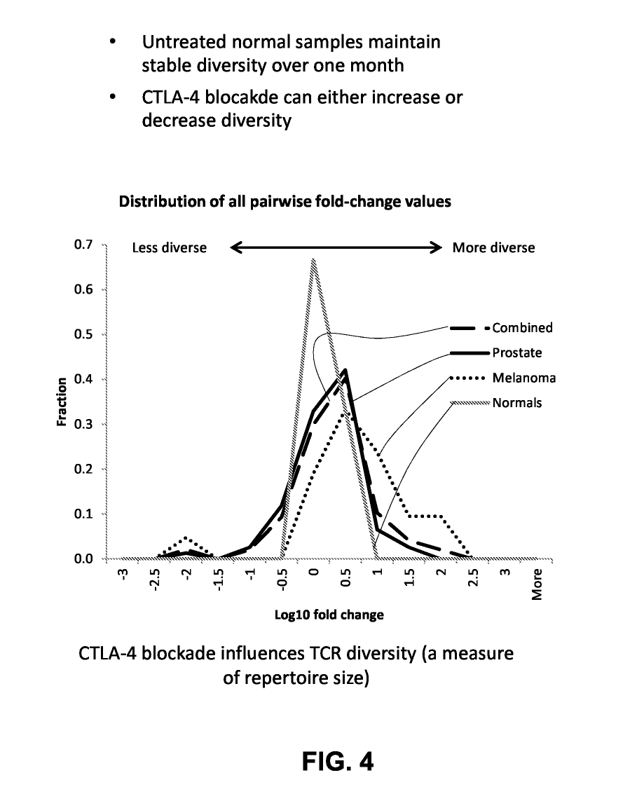

FIG. 4 illustrates data that shows the effect of CTLA-4 blockade on T cell repertoire diversity. To assess the degree of change, the difference between baseline and month 1 (post treatment) samples were quantified by applying Morisita's distance measure to clone count distributions, scaled from 0 to 1 to indicate minimal and maximal distance, respectively, e.g. Morisita, Mem. Fac. Sci. Kyushu Univ. Ser. E (Biol.) 3: 65-80 (1959). This metric is an inverse measure of overlap between two populations (baseline and 4 weeks after treatment). Whereas untreated subjects consistently showed the greatest overlap (minimal travel distance) in repertoires before and 4 weeks after treatment, both prostate cancer and melanoma patients showed a wide distribution of pairwise travel distance, extending out to maximum distance with respect to repertoire change. The median distance between untreated samples was 0.039 versus 0.197 for anti-CTLA-4 treated samples (P=0.0005, Mann-Whitney). These results indicate that anti-CTLA-4 monoclonal antibody treatment induces significant changes in clonotype frequencies consistent with T cell repertoire turnover. To assess whether anti-CTLA-4 influenced the diversity of the repertoire, the metric of repertoire size was used as a measure of sample diversity. The number of unique clonotypes was counted that were represented in the top 25th percentile by ranked molecule count after sorting by abundance. Fold-changes were then determined after first treatment. This metric is not strongly influenced by rare clonotypes and is therefore relatively stable to sequencing depth differences and different input cell amounts. The metric was calculated for paired pre- and post-treatment patient samples separated by one month, as well as untreated control samples, separated by the same time interval. In comparison with untreated subjects, who maintained stable diversity over one month, cancer patients treated with anti-CTLA-4 displayed increases in repertoire size beyond the range observed in untreated pairs (FIG. 4). 34 (45%) of 76 paired CRPC samples and 12 (57%) of 21 paired melanoma samples had >2-fold changes in TCR diversity. Overall, 46 (47%) of all 97 paired samples across prostate and melanoma patients had changes in diversity >2-fold in either direction. By comparison, none of the 9 untreated sample pairs underwent >2-fold change in diversity (P=0.005, Fisher's exact test, two-tailed). The greatest fold-increases were observed in melanoma patients, with 43% showing ≧10-fold increases, whereas 9% of all paired CRPC samples demonstrated similar changes (FIG. 4).

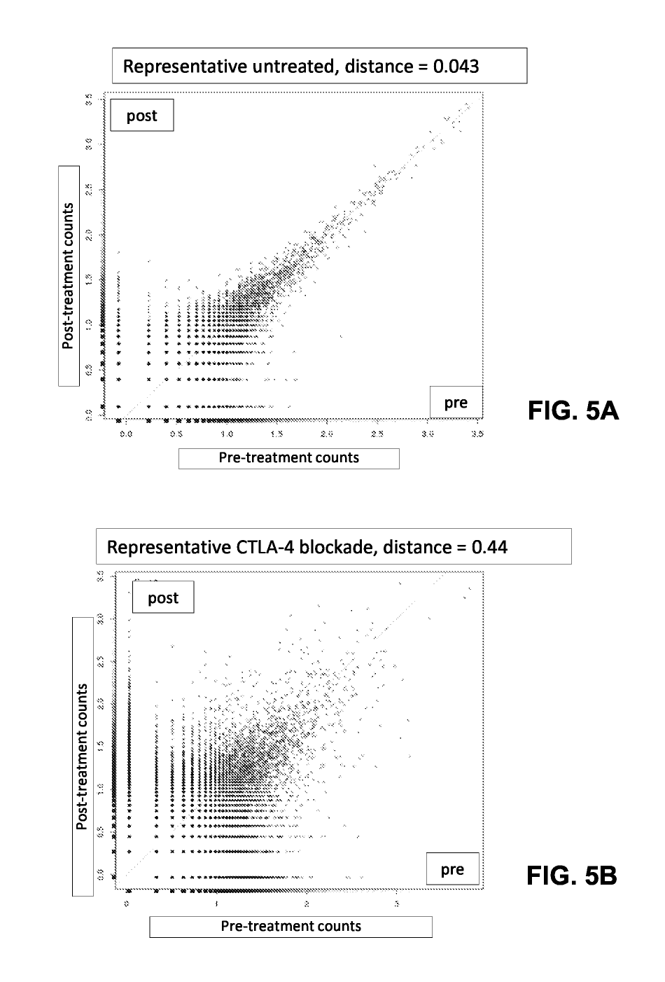

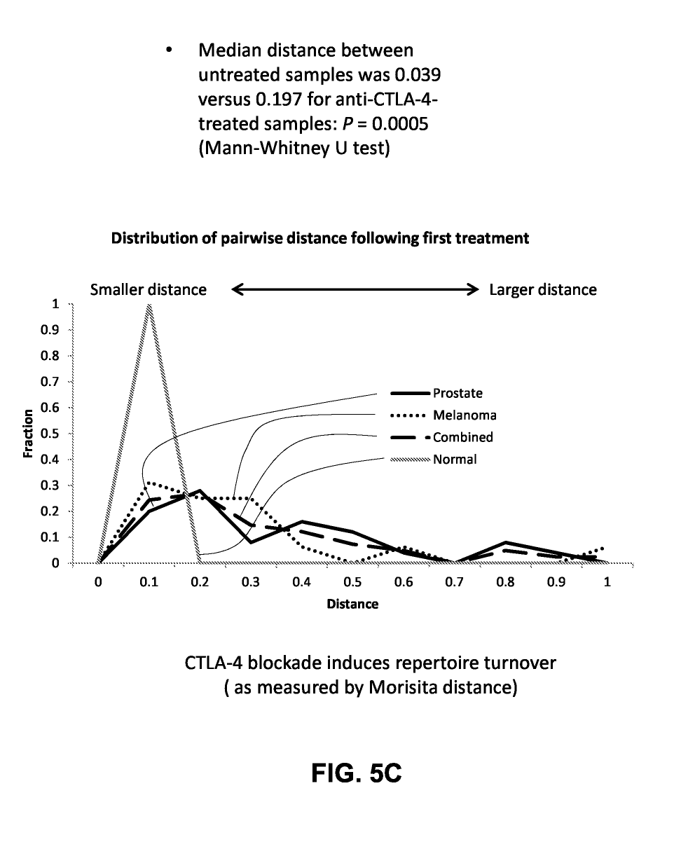

FIGS. 5A and 5B show data that further illustrate the effect of anti-CTLA-4 monoclonal antibody treatment on T cell repertoire turnover. FIG. 5A shows minimal variation in individual T cell clonotype abundance from month to month in untreated control subjects. In contrast, every patient who received at least one treatment of anti-CTLA-4 monoclonal antibody developed increases and decreases in absolute clonotype counts, as shown in FIG. 5B. In these figures, each point on the scatter plots represents a single clonotype with normalized log₁₀ clone count graphed at baseline (x axis) and after 1 month (y axis). The increased variance of the low-abundance clones is due to Poisson sampling effects.

FIG. 5C shows the difference between pre- and post-treatment samples (and untreated, sequential, normal samples) which was quantified by applying Morisita's distance measure to clone count distributions, with 0 indicating minimal distance and 1 indicating maximal distance.

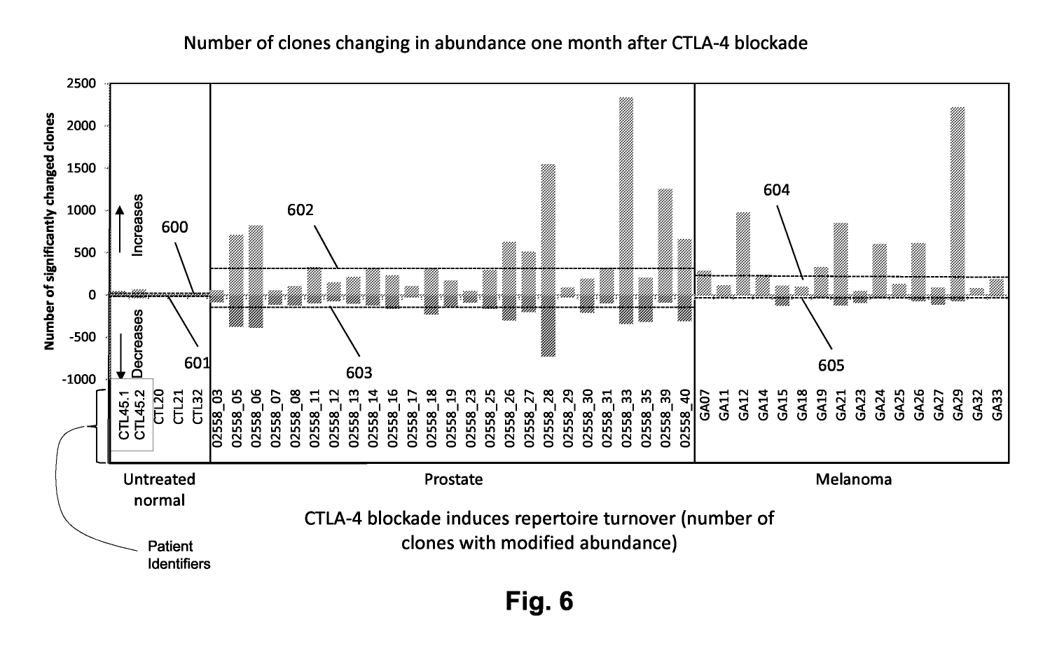

FIG. 6 shows data on the number of clones with significant abundance changes after treatment. The numbers of clones with significantly changed abundance one month after first treatment are plotted for each sample, with increased abundance clones plotted above the axis, and reduced abundance clones plotted as negative values. Median values for untreated control, prostate, and melanoma groups are plotted as dashed lines (600, increased untreated; 601, decreased untreated; 602, increased prostate; 603, decreased prostate; 604, increased melanoma; 605, decreased melanoma).

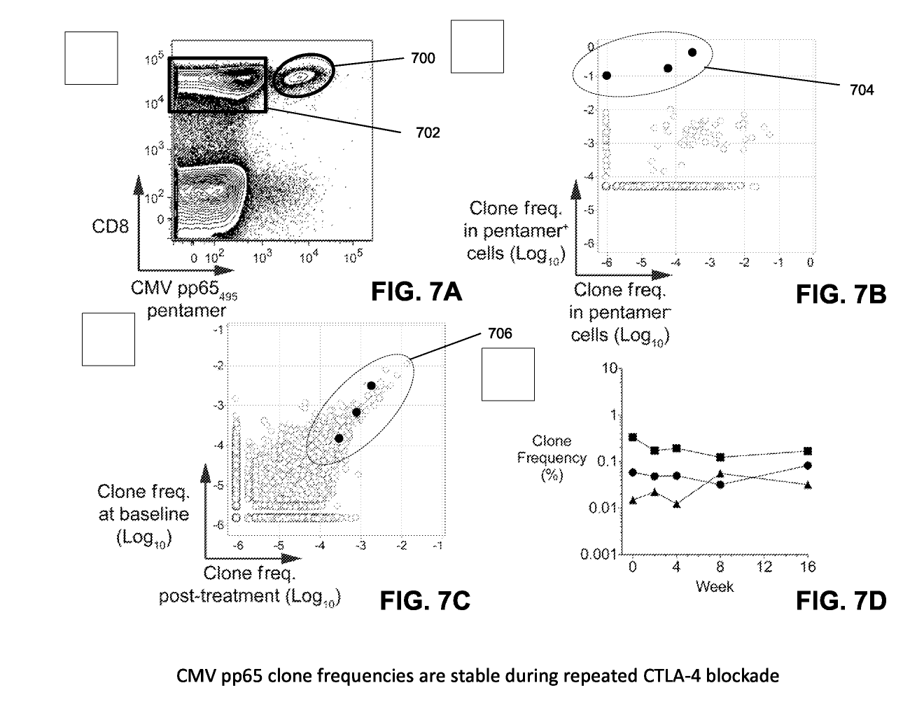

To further elucidate the nature of the pre-existing high frequency clonotypes on anti-CTLA-4 treatment, MHC/peptide tetramers were used to isolate and examine the evolution of T cell responses to specific antigens. Virus-specific T cells, which typically possess high affinity TCR, can be frequently identified with this approach. Indeed, CMV-reactive clones could be detected using HLA-A*0201/pp65 peptide tetramers (FIG. 7A). Tetramer+ and tetramer− CD8+ T cells were then sorted and sequenced for TCRβ VDJ regions (FIG. 7B) to identify the CMV pp65-specific TCRβ clones for specific patients. From one clinical responder with CRPC (partial response, 56 month survival), 3 unique clonotypes were identified that accounted for 80% of tetramer+ sorted cells (FIG. 7B). The overall frequency of these clonotypes was then assessed within the repertoire over time (FIG. 7C). At baseline, these 3 clonotypes were present at high frequencies (≧˜10⁻³) or at moderately high levels (10⁻⁴). Frequencies were stable after initial treatment (FIG. 7C) and maintained over the 4 cycles (FIG. 7D). The predicted CDR3 amino acid sequence from the most dominant clone matched a previously published pp65-specific clonotype (Roux et al, Clin. Immunology, 148: 16-26 (2013)) consistent with CMV-reactive repertoires dominated by a few shared clonotypes with high antigen affinity/avidity across MHC-matched individuals.

TABLE I
Data From Prostate Patients

| | Number of | Number of | | | | | |
|---|---|---|---|---|---|---|---|
| | Clonotypes | Clonotypes | | | | | |
| | with Increased | with Reduced | Morisita | | PFS* | OS | Survival |
| Patient | Abundance | Abundance | Value | Response | (mo) | (mo) | Group\hat{ } |
| 02558_25 | 297 | 165 | 0.475 | PD* | | 2 | Low |
| 02558_18 | 318 | 229 | 0.197 | PD | | 5 | Low |
| 02558_05 | 712 | 375 | 0.139 | PD | | 11 | Low |
| 02558_16 | 235 | 165 | 0.392 | PD | | 12 | Low |
| 02558_14 | 312 | 123 | 0.141 | PD | | 13 | Low |
| 02558_26 | 629 | 299 | 0.017 | PD | | 13 | Low |
| 02558_30 | 192 | 208 | 0.019 | PD | | 13 | Low |
| 02558_40 | 660 | 308 | 0.325 | PD | | 15 | Low |
| 02558_29 | 90 | 26 | 0.288 | PD | | 16 | Low |
| 02558_06 | 824 | 388 | 0.436 | PD | | 17 | Low |
| 02558_35 | 207 | 317 | 0.435 | PD | | 22 | Low |
| 02558_33 | 2341 | 342 | 0.856 | Responder | | 25 | Low |
| 02558_11 | 330 | 97 | 0.310 | PD | | 28 | High |
| 02558_27 | 513 | 202 | 0.736 | PD | | 29 | High |
| 02558_08 | 104 | 121 | 0.120 | PD | | 39 | High |
| 02558_28 | 1549 | 726 | 0.592 | PD | | 44 | High |
| 02558_39 | 1252 | 89 | 0.074 | PD | | 45 | High |
| 02558_07 | 58 | 114 | 0.400 | PD | | 47 | High |
| 02558_17 | 106 | 25 | 0.099 | PD | | 51 | High |
| 02558_23 | 47 | 89 | 0.165 | PD | | 60 | High |
| 02558_31 | 322 | 99 | 0.171 | PD | | 73 | High |
| 02558_12 | 152 | 73 | 0.113 | PD | | 82 | High |
| 02558_19 | 173 | 134 | 0.268 | Responder | | 96 | High |
| 02558_03 | 56 | 86 | 0.034 | PD | | 108 | High |
| 02558_13 | 213 | 103 | 0.701 | PD | | | High |

*“PD” means progressive disease; “PFS”means progression-free survival.
\hat{ }“Low” means post-treatment survival of less than 25 months; “High” means post-treatment survival of more than 25 months.

TABLE II
Data From Melanoma Patients

| | Number of | Number of | | | | | |
|---|---|---|---|---|---|---|---|
| | Clonotypes | Clonotypes | | | | | |
| | with | with | | | | | Sur- |
| | Increased | Reduced | Morisita | Re- | PFS | OS | vival |
| Patient | Abundance | Abundance | Value | sponse | (mo) | (mo) | Group |
| GA24 | 601 | 37 | 0.184 | PD | 2 | 3 | Low |
| GA23 | 49 | 93 | 0.115 | PD | 2 | 4 | Low |
| GA11 | 113 | 19 | 0.083 | PD | 2 | 7 | Low |
| GA21 | 850 | 121 | 0.034 | PD | 3 | 8 | Low |
| GA25 | 131 | 20 | 0.076 | PD | 3 | 8 | Low |
| GA27 | 88 | 113 | 0.219 | PD | 6 | 11 | Low |
| GA14 | 239 | 16 | 0.217 | PD | 3 | 15 | Low |
| GA15 | 109 | 128 | 0.243 | PD | 4 | 15 | Low |
| GA12 | 974 | 5 | 0.165 | PD | 2 | 20 | High |
| GA19 | 329 | 29 | 0.050 | PD | 3 | 36 | High |
| GA29 | 2221 | 71 | 0.520 | CR | 45 | 45 | High |
| GA26 | 616 | 73 | 0.974 | PD | 2 | 51 | High |
| GA33 | 187 | 14 | 0.312 | CR | 55 | 55 | High |
| GA32 | 83 | 4 | 0.246 | PD | 5 | 56 | High |
| GA18 | 99 | 9 | 0.060 | CR | 62 | 62 | High |
| GA07 | 288 | 1 | 0.166 | PD | 2 | 67 | High |

\hat{ }“Low” means post-treatment survival of less than 19 months; “High” means post-treatment survival of more than 19 months.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Aligning” means a method of comparing a test sequence, such as a sequence read, to one or more reference sequences to determine which reference sequence or which portion of a reference sequence is closest based on some sequence distance measure. An exemplary method of aligning nucleotide sequences is the Smith Waterman algorithm. Distance measures may include Hamming distance, Levenshtein distance, or the like. Distance measures may include a component related to the quality values of nucleotides of the sequences being compared.

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Antibody binding compound” means a compound derived from an antibody which compound is capable of specifically binding to a target molecule. Antibody binding compounds include, but are not limited to, antibody fragments, such as Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

“Clonotype” means a recombined nucleotide sequence of a lymphocyte which encodes an immune receptor or a portion thereof, such as a CDR3 region. More particularly, clonotype means a recombined nucleotide sequence of a T cell or B cell which encodes a T cell receptor (TCR) chain or B cell receptor (BCR) chain, or a portion thereof. In various embodiments, clonotypes may encode all or a portion of a VDJ rearrangement of IgH, a DJ rearrangement of IgH, a VJ rearrangement of IgK, a VJ rearrangement of IgL, a VDJ rearrangement of TCR β, a DJ rearrangement of TCR β, a VJ rearrangement of TCR α, a VJ rearrangement of TCR γ, a VDJ rearrangement of TCR δ, a VD rearrangement of TCR δ, a Kde-V rearrangement, or the like. Clonotypes may also encode translocation breakpoint regions involving immune receptor genes, such as Bcl1-IgH or Bcl1-IgH. In one aspect, clonotypes have sequences that are sufficiently long to represent or reflect the diversity of the immune molecules that they are derived from; consequently, clonotypes may vary widely in length. In some embodiments, clonotypes have lengths in the range of from 25 to 400 nucleotides; in other embodiments, clonotypes have lengths in the range of from 25 to 200 nucleotides.

“Clonotype profile” means a listing of distinct clonotypes and their relative abundances that are derived from a population of lymphocytes. Typically, the population of lymphocytes are obtained from a tissue sample. The term “clonotype profile” is related to, but more general than, the immunology concept of immune “repertoire” as described in references, such as the following: Arstila et al, Science, 286: 958-961 (1999); Yassai et al, Immunogenetics, 61: 493-502 (2009); Kedzierska et al, Mol. Immunol., 45(3): 607-618 (2008); and the like. The term “clonotype profile” includes a wide variety of lists and abundances of rearranged immune receptor-encoding nucleic acids, which may be derived from selected subsets of lymphocytes (e.g. tissue-infiltrating lymphocytes, immunophenotypic subsets, or the like), or which may encode portions of immune receptors that have reduced diversity as compared to full immune receptors. In some embodiments, clonotype profiles may comprise at least 10³ distinct clonotypes; in other embodiments, clonotype profiles may comprise at least 10⁴ distinct clonotypes; in other embodiments, clonotype profiles may comprise at least 10⁵ distinct clonotypes; in other embodiments, clonotype profiles may comprise at least 10⁶ distinct clonotypes. In such embodiments, such clonotype profiles may further comprise abundances or relative frequencies of each of the distinct clonotypes. In one aspect, a clonotype profile is a set of distinct recombined nucleotide sequences (with their abundances) that encode T cell receptors (TCRs) or B cell receptors (BCRs), or fragments thereof, respectively, in a population of lymphocytes of an individual, wherein the nucleotide sequences of the set have a one-to-one correspondence with distinct lymphocytes or their clonal subpopulations for substantially all of the lymphocytes of the population. In one aspect, nucleic acid segments defining clonotypes are selected so that their diversity (i.e. the number of distinct nucleic acid sequences in the set) is large enough so that substantially every T cell or B cell or clone thereof in an individual carries a unique nucleic acid sequence of such repertoire. That is, preferably each different clone of a sample has different clonotype. In other aspects of the invention, the population of lymphocytes corresponding to a repertoire may be circulating B cells, or may be circulating T cells, or may be subpopulations of either of the foregoing populations, including but not limited to, CD4+ T cells, or CD8+ T cells, or other subpopulations defined by cell surface markers, or the like. Such subpopulations may be acquired by taking samples from particular tissues, e.g. bone marrow, or lymph nodes, or the like, or by sorting or enriching cells from a sample (such as peripheral blood) based on one or more cell surface markers, size, morphology, or the like. In still other aspects, the population of lymphocytes corresponding to a repertoire may be derived from disease tissues, such as a tumor tissue, an infected tissue, or the like. In one embodiment, a clonotype profile comprising human TCR β chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶. In another embodiment, a clonotype profile comprising human IgH chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶. In a particular embodiment, a clonotype profile of the invention comprises a set of nucleotide sequences encoding substantially all segments of the V(D)J region of an IgH chain. In one aspect, “substantially all” as used herein means every segment having a relative abundance of 0.001 percent or higher; or in another aspect, “substantially all” as used herein means every segment having a relative abundance of 0.0001 percent or higher. In another particular embodiment, a clonotype profile of the invention comprises a set of nucleotide sequences that encodes substantially all segments of the V(D)J region of a TCR β chain. In another embodiment, a clonotype profile of the invention comprises a set of nucleotide sequences having lengths in the range of from 25-200 nucleotides and including segments of the V, D, and J regions of a TCR β chain. In another embodiment, a clonotype profile of the invention comprises a set of nucleotide sequences having lengths in the range of from 25-200 nucleotides and including segments of the V, D, and J regions of an IgH chain. In another embodiment, a clonotype profile of the invention comprises a number of distinct nucleotide sequences that is substantially equivalent to the number of lymphocytes expressing a distinct IgH chain. In another embodiment, a clonotype profile of the invention comprises a number of distinct nucleotide sequences that is substantially equivalent to the number of lymphocytes expressing a distinct TCR β chain. In still another embodiment, “substantially equivalent” means that with ninety-nine percent probability a clonotype profile will include a nucleotide sequence encoding an IgH or TCR 0 or portion thereof carried or expressed by every lymphocyte of a population of an individual at a frequency of 0.001 percent or greater. In still another embodiment, “substantially equivalent” means that with ninety-nine percent probability a repertoire of nucleotide sequences will include a nucleotide sequence encoding an IgH or TCR 0 or portion thereof carried or expressed by every lymphocyte present at a frequency of 0.0001 percent or greater. In some embodiments, clonotype profiles are derived from samples comprising from 10⁵ to 10⁷ lymphocytes. Such numbers of lymphocytes may be obtained from peripheral blood samples of from 1-10 mL.

“Complementarity determining regions” (CDRs) mean regions of an immunoglobulin (i.e., antibody) or T cell receptor where the molecule complements an antigen's conformation, thereby determining the molecule's specificity and contact with a specific antigen. T cell receptors and immunoglobulins each have three CDRs: CDR1 and CDR2 are found in the variable (V) domain, and CDR3 includes some of V, all of diverse (D) (heavy chains only) and joint (J), and some of the constant (C) domains.

“CTLA-4 inhibitor” means a compound that specifically binds to the extracellular domain of CTLA-4 and blocks the binding of CTLA-4 to CD80 or CD86. In some embodiments, a CTLA-4 inhibitor comprises an antibody binding compound, such as an antibody or an antigen-binding fragment thereof. U.S. Pat. Nos. 5,855,887; 5,811,097; 6,682,736; 7,452,535 disclose antibodies specific for human CTLA-4, including antibodies specific for the extracellular domain of CTLA-4 and which are capable of blocking its binding to CD80 or CD86; methods of making such antibodies, and methods of using such antibodies as anti-cancer agents; accordingly such patents are incorporated herein by reference.

“Lymphoid or myeloid proliferative disorder” means any abnormal proliferative disorder in which one or more nucleotide sequences encoding one or more rearranged immune receptors can be used as a marker for monitoring such disorder. “Lymphoid or myeloid neoplasm” means an abnormal proliferation of lymphocytes or myeloid cells that may be malignant or non-malignant. A lymphoid cancer is a malignant lymphoid neoplasm. A myeloid cancer is a malignant myeloid neoplasm. Lymphoid and myeloid neoplasms are the result of, or are associated with, lymphoproliferative or myeloproliferative disorders, and include, but are not limited to, follicular lymphoma, chronic lymphocytic leukemia (CLL), acute lymphocytic leukemia (ALL), chronic myelogenous leukemia (CML), acute myelogenous leukemia (AML), Hodgkins's and non-Hodgkin's lymphomas, multiple myeloma (MM), monoclonal gammopathy of undetermined significance (MGUS), mantle cell lymphoma (MCL), diffuse large B cell lymphoma (DLBCL), myelodysplastic syndromes (MDS), T cell lymphoma, or the like, e.g. Jaffe et al, Blood, 112: 4384-4399 (2008); Swerdlow et al, WHO Classification of Tumours of Haematopoietic and Lymphoid Tissues (e. 4th) (IARC Press, 2008).

“Percent homologous,” “percent identical,” or like terms used in reference to the comparison of a reference sequence and another sequence (“comparison sequence”) mean that in an optimal alignment between the two sequences, the comparison sequence is identical to the reference sequence in a number of subunit positions equivalent to the indicated percentage, the subunits being nucleotides for polynucleotide comparisons or amino acids for polypeptide comparisons. As used herein, an “optimal alignment” of sequences being compared is one that maximizes matches between subunits and minimizes the number of gaps employed in constructing an alignment. Percent identities may be determined with commercially available implementations of algorithms, such as that described by Needleman and Wunsch, J. Mol. Biol., 48: 443-453 (1970)(“GAP” program of Wisconsin Sequence Analysis Package, Genetics Computer Group, Madison, Wis.), or the like. Other software packages in the art for constructing alignments and calculating percentage identity or other measures of similarity include the “BestFit” program, based on the algorithm of Smith and Waterman, Advances in Applied Mathematics, 2: 482-489 (1981) (Wisconsin Sequence Analysis Package, Genetics Computer Group, Madison, Wis.). In other words, for example, to obtain a polynucleotide having a nucleotide sequence at least 95 percent identical to a reference nucleotide sequence, up to five percent of the nucleotides in the reference sequence may be deleted or substituted with another nucleotide, or a number of nucleotides up to five percent of the total number of nucleotides in the reference sequence may be inserted into the reference sequence.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Quality score” means a measure of the probability that a base assignment at a particular sequence location is correct. A variety methods are well known to those of ordinary skill for calculating quality scores for particular circumstances, such as, for bases called as a result of different sequencing chemistries, detection systems, base-calling algorithms, and so on. Generally, quality score values are monotonically related to probabilities of correct base calling. For example, a quality score, or Q, of 10 may mean that there is a 90 percent chance that a base is called correctly, a Q of 20 may mean that there is a 99 percent chance that a base is called correctly, and so on. For some sequencing platforms, particularly those using sequencing-by-synthesis chemistries, average quality scores decrease as a function of sequence read length, so that quality scores at the beginning of a sequence read are higher than those at the end of a sequence read, such declines being due to phenomena such as incomplete extensions, carry forward extensions, loss of template, loss of polymerase, capping failures, deprotection failures, and the like.

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

What is claimed is:

1. A method of predicting clinical response of a patient to treatment of a cancer by an immune checkpoint pathway inhibitor, the method comprising the steps of:
(a) generating a first clonotype profile from recombined T cell receptor genes or nucleic acids transcribed therefrom from a first patient sample taken before treatment by an immune checkpoint pathway inhibitor;
(b) generating a second clonotype profile from recombined T cell receptor genes or nucleic acids transcribed therefrom from a second patient sample taken during or after treatment by an immune checkpoint pathway inhibitor;
(c) determining a number of clonotypes that decrease in frequency between the first and second clonotype profiles; and
(d) predicting a lack of responsiveness in the patient to the treatment whenever the number of clonotypes that decrease in frequency is greater than a predetermined value.

2. The method of claim 1 wherein clonotype frequencies of said first clonotype profile greater than 10⁻⁶ form a baseline set of clonotypes that are compared to clonotype frequencies of a clonotype profile of a successive sample.

3. The method of claim 2 wherein said predetermined value is a number of clonotypes in said baseline set in the range of from 10 to 1000.

4. The method of claim 2 wherein said predetermined value is a number corresponding to at least twenty-five percent of said baseline set.

5. The method of claim 1 wherein said immune checkpoint pathway inhibitor is CTLA-4 or PD-1.

6. The method of claim 5 wherein said immune checkpoint pathway inhibitor is CTLA-4.

7. A method of predicting clinical response of a patient to treatment of a cancer by a CTLA-4 inhibitor, the method comprising the steps of:
(a) generating a first clonotype profile from recombined T cell receptor genes or nucleic acids transcribed therefrom from a first patient sample taken before treatment by a CTLA-4 inhibitor;
(b) generating a second clonotype profile from recombined T cell receptor genes or nucleic acids transcribed therefrom from a second patient sample taken during or after treatment by a CTLA-4 inhibitor;
(c) determining a number of clonotypes that decrease in frequency between the first and second clonotype profiles; and
(d) predicting a lack of responsiveness in the patient to the treatment whenever the number of clonotypes that decrease in frequency is greater than a predetermined value.

8. The method of claim 7 wherein each of said first and second clonotype profiles comprise at least 10³ clonotypes and said predetermined value is in a range of from 10 to 1000 clonotypes.

9. The method of claim 7 wherein said decrease in frequency is any statistically significant decrease.

10. The method of claim 7 wherein said number is based on decreases in frequencies of clonotypes that each have a frequency in said first clonotype profile of at least 10⁻⁵.

11. The method of claim 7 wherein said decrease in frequency is at least a two-fold decrease.

12. The method of claim 7 wherein said first patient sample is taken from said patient within one week prior to initiation of said treatment.

13. The method of claim 12 wherein said first patient sample is taken at the time treatment is initiated.

14. The method of claim 7 wherein said second patient sample is taken from said patient within three months after initiation of said treatment.

15. The method of claim 14 wherein said second patient sample is taken from said patient within one month after initiation of said treatment.

16. The method of claim 7 wherein said CTLA-4 inhibitor is a therapeutic antibody.

17. The method of claim 16 wherein said therapeutic antibody is ipilimumab or tremelimumab or an antibody binding compound derived therefrom.

18. The method of claim 7 wherein said cancer is a prostate cancer or a melanoma.

19. A method of selecting a patient having a cancer for treatment by a CTLA-4 inhibitor, the method comprising the steps of:
(a) generating a first clonotype profile from recombined T cell receptor genes or nucleic acids transcribed therefrom from a first patient sample taken before treatment by a CTLA-4 inhibitor;
(b) generating a second clonotype profile from recombined T cell receptor genes or nucleic acids transcribed therefrom from a second patient sample taken during or after treatment by a CTLA-4 inhibitor;
(c) determining a number of clonotypes that decrease in frequency between the first and second clonotype profiles; and
(d) selecting the patient for treatment with a CTLA-4 inhibitor whenever the number of clonotypes that decrease in frequency is below a predetermined value.

20. The method of claim 19 wherein clonotype frequencies of said first clonotype profile greater than 10⁻⁶ form a baseline set of clonotypes that are compared to clonotype frequencies of a clonotype profile of a successive sample.
